(19) United States
(12) Patent Application Publication
(10) Pub. No.: US 2015/0100006 A1
(43) Pub. Date: Apr. 9, 2015

(54) ANGLE-ADJUSTABLE REHABILITATION DEVICE

(71) Applicant: Ming-Chieh Lu, Miaoli County (TW)

(72) Inventor: Ming-Chieh Lu, Miaoli County (TW)

(21) Appl. No.: 14/299,562

(22) Filed: Jun. 9, 2014

Related U.S. Application Data
(63) Continuation-in-part of application No. 14/132,344, filed on Dec. 18, 2013.

(30) Foreign Application Priority Data
Oct. 3, 2013 (TW) 102218504

(51) Int. Cl. A61F 5/01

(52) U.S. Cl. CPC A61F 5/0102; A61F 2005/0132
USPC 602/16

(57) ABSTRACT

An angle-adjustable rehabilitation device includes first and second racks between two lateral covers, a locking unit of the second rack is capable of switching the first rack between an adjustable position and a nonadjustable position. When in the adjustable position, the rotatable angle of the first rack can be freely adjusted. The control members of the two angle-adjustment positioning members protrude out of the two lateral covers, therefore, angle adjustment can be easily done without removing any components. Furthermore, the arc-shaped angle scale on the two lateral covers makes the angle adjustment more readable and controllable, and the first rack can be moved back into the non-adjustable position automatically after angle adjustment is done.

This application is a continuation in part of U.S. patent application Ser. No. 14/132,344, which claims the benefit of the earlier filing date of Dec. 18, 2013.

### BACKGROUND OF THE INVENTION

1. Field of the Invention

The present invention relates to a rehabilitation device, and more particularly to an angle-adjustable rehabilitation device.

2. Description of the Prior Art

Human body motion is generally conducted through the joints of arms and legs, and once the joints are injured, a good rehabilitation device would become very important for rehabilitation of the injured joints.

Conventionally, rehabilitation devices are mostly provided with a plurality of pivoting elements, return members and positioning members between the cover and the second pivoting member, in order to provide limited adjustment of the rotation angle of the joints. Therefore, in addition to the disadvantages of complicated structure and assembly process, the cover of conventional rehabilitation devices has to be removed, otherwise angle adjustment cannot be adjusted easily and swayed easily due to parts tolerance and wear after use.

The present invention has arisen to mitigate and/or obviate the afore-described disadvantages.

### SUMMARY OF THE INVENTION

The primary objective of the present invention is to provide an angle-adjustable rehabilitation device, wherein the angle can be adjusted without removing any components of the rehabilitation device, and the respective components can be positioned firmly during angle adjustment.

To achieve the above objective, an angle-adjustable rehabilitation device in accordance with the present invention comprises:

two lateral covers each including a cover member, each of the cover members including at least one angle-adjustment section which consists of a plurality of positioning notches defined on an arc-shaped slide section in a spaced manner, the positioning notches being in communication with the slide section, each of the toothed discs being provided with an elongated slot, the cover member of one of the two lateral covers being formed with an operation slot in communication with the elongated slot;

between the two lateral covers being disposed two angle-adjustment positioning members, a first rack and a second racks in such a manner that the two cover members are oppositely arranged to define therebetween a lateral slot, between the two lateral covers and the first and second racks being disposed a flexible member to reduce friction, improve buffering effect and enable the two lateral covers and the first and second racks to be abutted more closely against one another;

each of the two angle-adjustment positioning members including a main block, a control member, a connecting member and two springs; wherein:

the main block is formed with a cavity and a slide groove in communication with the cavity;

the control member is partially disposed in the cavity;

the connecting member is interactively connected to the control member and extends out of the slide groove, each of the springs is disposed between the control member and a bottom of the cavity;

on each of two opposite outer surfaces of the main block is provided an arc-shaped slide block which is located between the cover members and slidably disposed in the slide section, the control member of each of the angle-adjustment positioning members partially extends out of the lateral slot, and the springs are pressed against the control members to maintain the connecting member at a position aligned to the positioning notches;

the first rack includes a first connecting section and a first extension section extending from the first connecting section, on the first connecting section is formed a plurality of positioning teeth, the first connecting section is pivotally disposed between the two lateral covers, and the first extension section extends out of the lateral slots;

the second rack is assembled to a locking unit and then disposed between the two lateral covers, the second rack includes a second connecting section and a second extension section extends from the second connecting section, the second connecting section is formed with an assembling slot; and

the locking unit includes an lubricating and positioning member and a control member; wherein:

the lubricating and positioning member are disposed the assembling slot, on an inner peripheral surface of the lubricating and positioning member are formed a first restricting notch and a second restricting notch;

the control member has one end formed with an engaging protrusion, and another formed with a plurality of engaging teeth, on one lateral surface of the control member is formed a control protrusion, the control member is abutted against the inner peripheral surface of the lubricating and positioning member in such a manner that the control protrusion of the control member is disposed in the first restricting notch or the second restricting notch of the elastic stop member, the lubricating and positioning member allows the control member to slide and to be fixed at different positions, the engaging teeth are engaged with the positioning teeth of the first rack, and the control protrusion is inserted through the elongated slot and extends out of the operation slot;

when the engaging protrusion is engaged in the first restricting notch, the engaging teeth of the control member are located in an open end of the assembling slot, and when the engaging protrusion is located within the second restricting notch, the engaging teeth of the control member extend out of the open end of the assembling slot and engage with the positioning teeth of the first rack.

### BRIEF DESCRIPTION OF THE DRAWINGS

FIG. 1 is a perspective view of an angle-adjustable rehabilitation device in accordance with the present invention;

FIG. 2 is an exploded view of the angle-adjustable rehabilitation device in accordance with the present invention;

FIG. 3 is an exploded view of an angle-adjustable rehabilitation device in accordance with another embodiment of the present invention;

FIG. 4 shows that the angle-adjustable rehabilitation device in accordance with the present invention is worn on a wearer;

FIG. 5 is a side view showing that the racks of the angle-adjustable rehabilitation device in accordance with the present invention are in a nonrotatable position;

FIG. 6 is a side view showing that the racks of the angle-adjustable rehabilitation device in accordance with the present invention are in a rotatable position;

FIG. 7 is a side view showing that the racks of the angle-adjustable rehabilitation device in accordance with the present invention are in a rotatable position, and the angle-adjustment positioning members are being pressed down;

FIG. 8 is an enlarged view of a part of FIG. 7;

FIG. 9 is an enlarged view showing that the racks of the angle-adjustable rehabilitation device in accordance with the present invention are in a rotatable position, and the angle-adjustment positioning members are being pressed down;

FIG. 10 is an enlarged view showing that the racks of the angle-adjustable rehabilitation device in accordance with the present invention are in a nonadjustable position;

FIG. 11 is a transparent view of a part of FIG. 10; and

FIG. 12 shows that the racks of the angle-adjustable rehabilitation device in accordance with the present invention are in a nonadjustable position.

### DETAILED DESCRIPTION OF THE PREFERRED EMBODIMENTS

The present invention will be clearer from the following description when viewed together with the accompanying drawings, which show, for purpose of illustrations only, the preferred embodiment in accordance with the present invention.

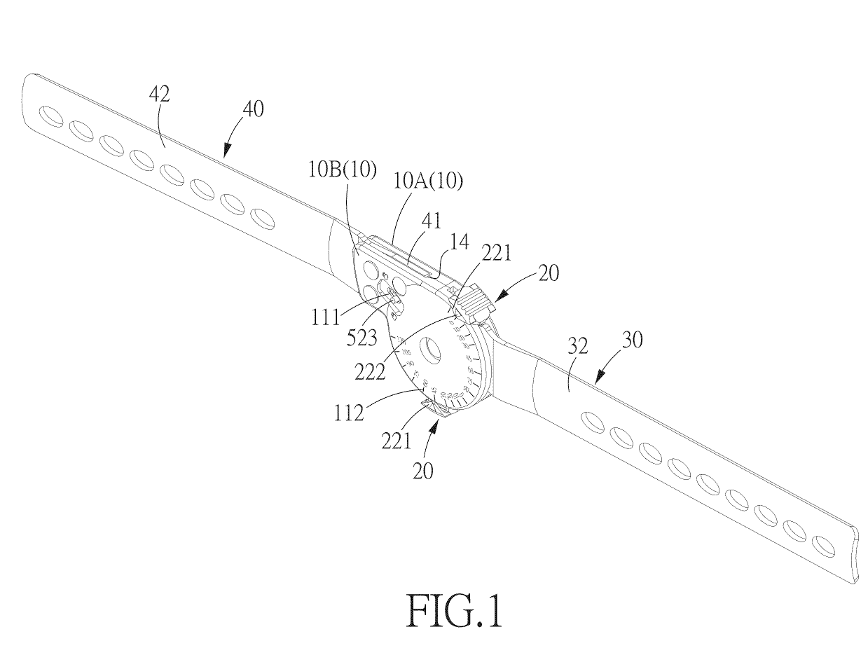

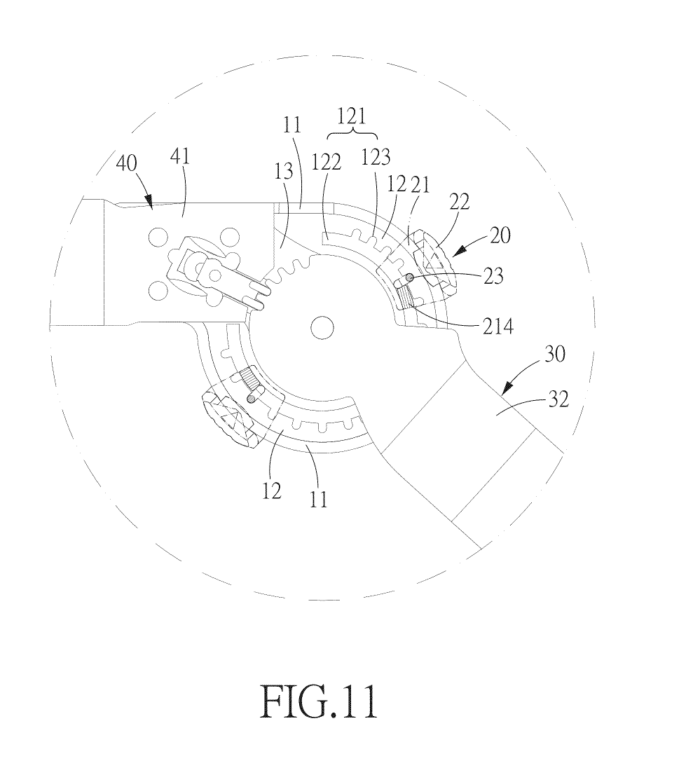

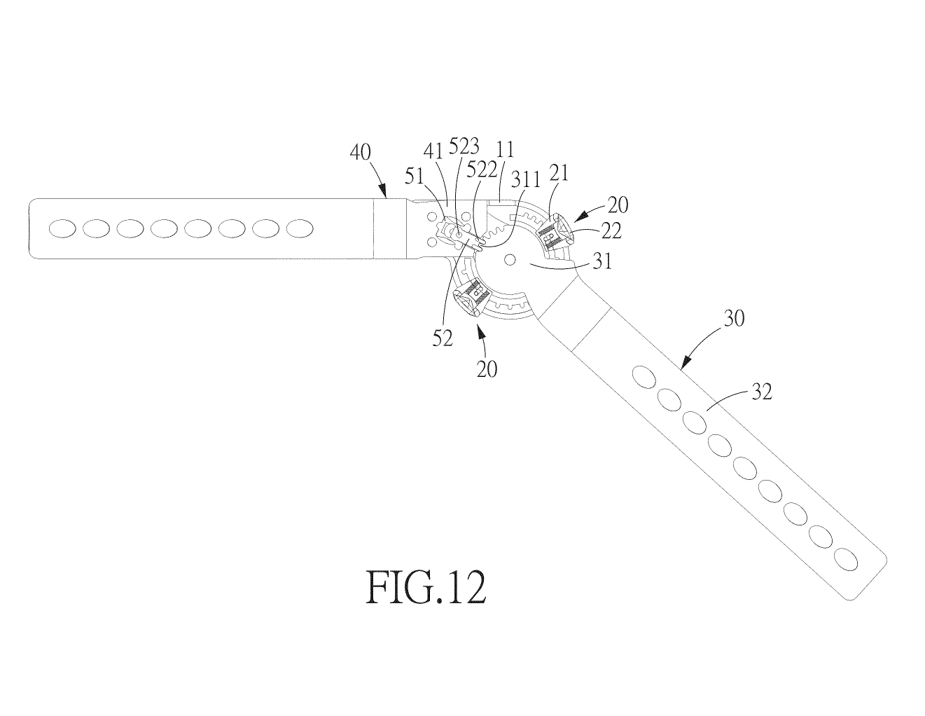

Referring to FIGS. 1-12, an angle-adjustable rehabilitation device in accordance with a preferred embodiment of the present invention comprises: two lateral covers 10, two angle-adjustment positioning members 20, a first rack 30, a second rack 40, and a locking unit 50.

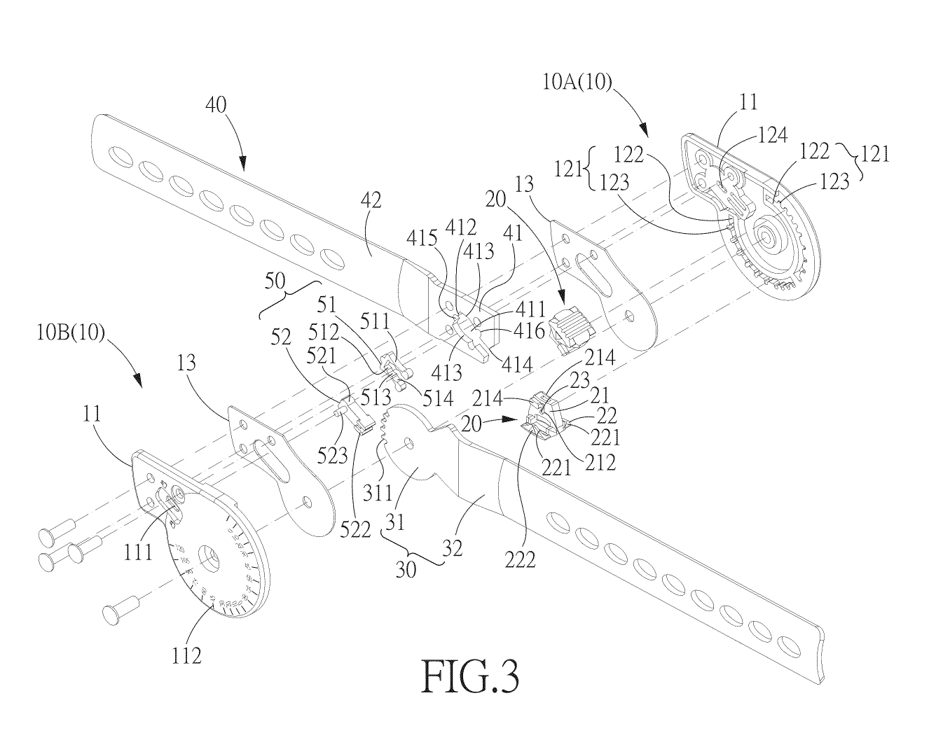

The two lateral covers 10 include a first lateral cover 10A and a second lateral cover 10B, and each include a cover member 11. On an inner surface of the cover member 11 is provided at least one angle-adjustment section 121 which consists of a plurality of positioning notches 123 defined on a slide section 122 in a spaced manner. The slide section 122 is arc-shaped, and the positioning notches 123 are in communication with the slide section 122. In this embodiment, on the inner surface of the cover member 11 are integrally formed two angle-adjustment sections 121 and an elongated slot 124, as shown FIG. 3.

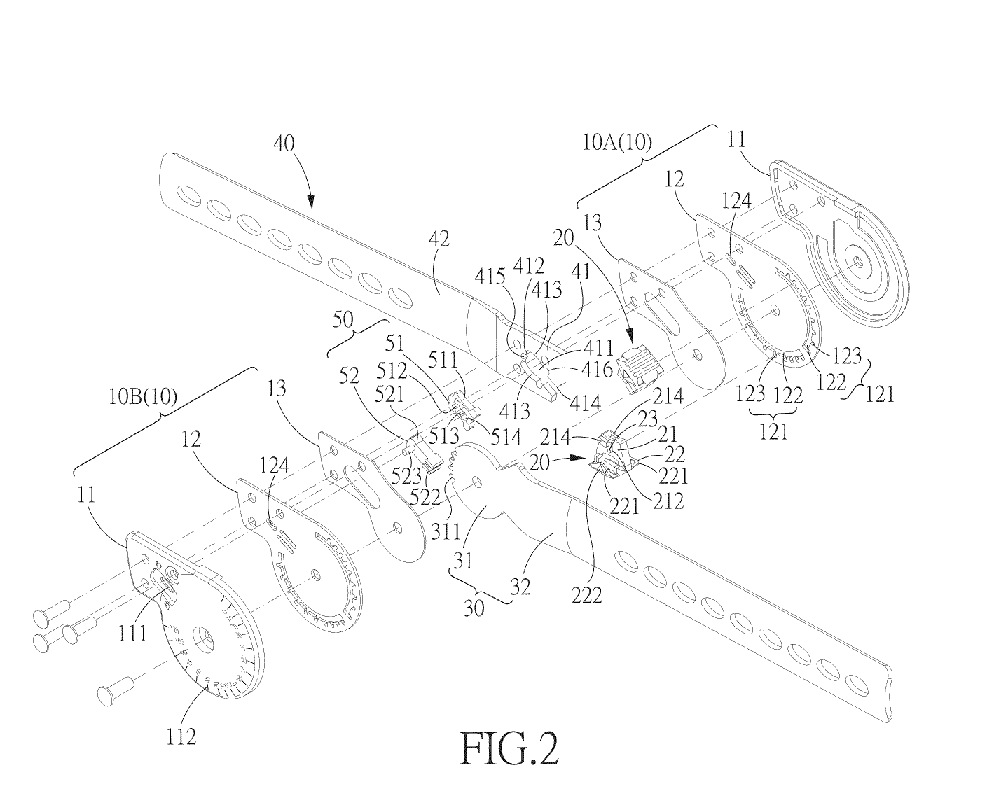

On the inner surface of the cover member 11 can also be provided a toothed disc 12, and the angle-adjustment sections 121 and the elongated slot 124 can be provided on the toothed disc 12, as shown in FIG. 2. The cover member 11 of the second lateral cover 10B is formed with an operation slot 111 in communication with the elongated slot 124 of the toothed disc 12, and on the outer surface of the cover member 11 of the second lateral cover 10B is formed an arc-shaped angle scale 112. The two angle-adjustment positioning members 20, the first and second racks, 30, 40 are disposed between the two lateral covers 10 in such a manner that the two cover members 11 are oppositely arranged to define therebetween a lateral slot 14. Between the two lateral covers 10 and the first and second racks, 30, 40 is disposed a buffer means to reduce friction and wear, improve buffering effect and enable the two lateral covers 10 and the first and second racks, 30, 40 to be abutted more closely against one another.

The buffer means can be a separator 13 which is made of a material softer than the first and second racks 30, 40, the cover members 11 and the toothed disc 12. In this embodiment, the first and second racks 30, 40, the cover members 11 and the toothed disc 12 are made of metal, and the separator 13 is made of plastic or rubber and has a lubrication function. When the angle-adjustment section 121 and the elongated slot 124 are integrally formed on the cover members 11, the separator 13 is disposed between the cover members 11 and the first and second racks 30, 40. When the angle-adjustment section 121 and the elongated slot 124 are formed on the toothed disc 12, the separator 13 is disposed between the toothed disc 12 and the first and second racks 30, 40. The soft separator 13 enables the adjacent components to be abutted against one another more closely, so as to reduce swing, friction and improve buffering effect, and also facilitate the angle adjustment.

Each of the two angle-adjustment positioning members 20 includes a main block 21, a control member 22, a connecting member 23 and two springs 24. The main block 21 is formed with a cavity 211 and a slide groove 212 in communication with the cavity 211. The control member 22 is partially disposed in the cavity 211, and the part of the control member 22 that protrudes out of the cavity 211 extends laterally in opposite directions to form two L-shaped retaining portions 221. On each of the retaining portions 221 is formed an indication portion 222 which is a triangular structure used as a mark for indicating the angle of adjustment. The connecting member 23 is interactively connected to the control member 22 and extends out of the slide groove 212. Each of the springs 24 is disposed between the control member 22 and a bottom 213 of the cavity 211, so that the control member 22 is able to elastically move within the main block 21. On each of two opposite outer surfaces of the main block 21 is provided an arc-shaped slide block 214 which is located between the cover members 11. The slide block 214 of each of the angle-adjustment positioning members 20 is slidably disposed in the slide section 122 of each of the toothed discs 12 of the lateral covers 10, the control member 22 of each of the angle-adjustment positioning members 20 partially extends out of the lateral slots 14, and the retaining portions 221 are located outside the lateral covers 10 and abutted against the cover members 11. The indication portion 222 of the retaining portions 221 points toward the arc-shaped angle scale 112. Each of the springs 24 is pressed against the corresponding control members 22 to maintain the connecting member 23 at a position aligned to the positioning notches 123.

The first rack 30 includes a first connecting section 31 and a first extension section 32 extending from the first connecting section 31. On the first connecting section 31 is formed a plurality of positioning teeth 311. The first connecting section 31 is pivotally disposed between the two lateral covers 10 and abutted against the separators 13. The first extension section 32 extends out of the lateral slot 14.

The second rack 40 is assembled to the locking unit 50 and then disposed between the two lateral covers 10 and abutted against the separators 13.

The second rack 40 includes a second connecting section 41 and a second extension section 42 extending from the second connecting section 41. The second connecting section 41 is formed with an assembling slot 411 which includes a close end 412, two opposite walls 413 and an open end 414. At the close end 412 is formed a first positioning notch 415, and on the opposite walls 413 are formed two second positioning notches 416. The second connecting section 41 is abutted against the separators 13.

The locking unit 50 includes a lubricating and positioning member 51 made of plastic material and a control member 52. An outer peripheral surface 511 of the lubricating and positioning member 51 is formed in the shape of the assembling slot 411, so as to make the lubricating and positioning member 51 fit in the assembling slot 411. On an inner peripheral surface 512 of the lubricating and positioning member 51 are formed two opposite first restricting notches 513 and two opposite second restricting notches 514. The control member 52 has one end formed with two opposite engaging protrusions 521, and another formed with a plurality of engaging teeth 522. On one lateral surface of the control member 52 is formed a cylindrical control protrusion 523. The control member 52 is abutted against the inner peripheral surface 512 of the lubricating and positioning member 51 in such a manner that the control protrusion 523 of the control member 52 is disposed in the first restricting notches 513 or the second restricting notches 514 of the lubricating and positioning member 51, the lubricating and positioning member 51 allows the control member 52 to slide and to be fixed at different positions, the engaging teeth 522 are engaged with the positioning teeth 311 of the first rack 30, and the control protrusion 523 is inserted through the elongated slot 124 of the second lateral cover 10B and extends out of the operation slot 111 of the second lateral cover 10B. When the engaging protrusions 521 are engaged in the first restricting notches 513, the engaging teeth 522 of the control member 52 will be located in the open end 414 of the assembling slot 411, and when the engaging protrusions 521 are located within the second restricting notches 514, the engaging teeth 522 of the control member 52 will extend out of the open end 414 of the assembling slot 411 and engage with the positioning teeth 311 of the first rack 30.

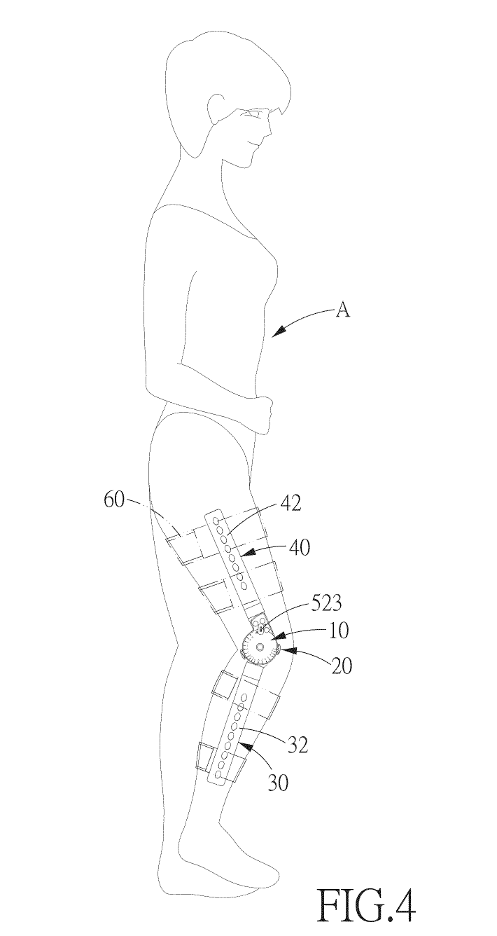

The angle-adjustable rehabilitation device of the present invention can be worn on the arms and legs of a wearer A by a fastening band 60, in such a manner that the lateral covers 10 are located the joints, and the first and second racks 30, 40 are fastened to the parts of the arms or legs adjacent to the joints, as shown in FIG. 4.

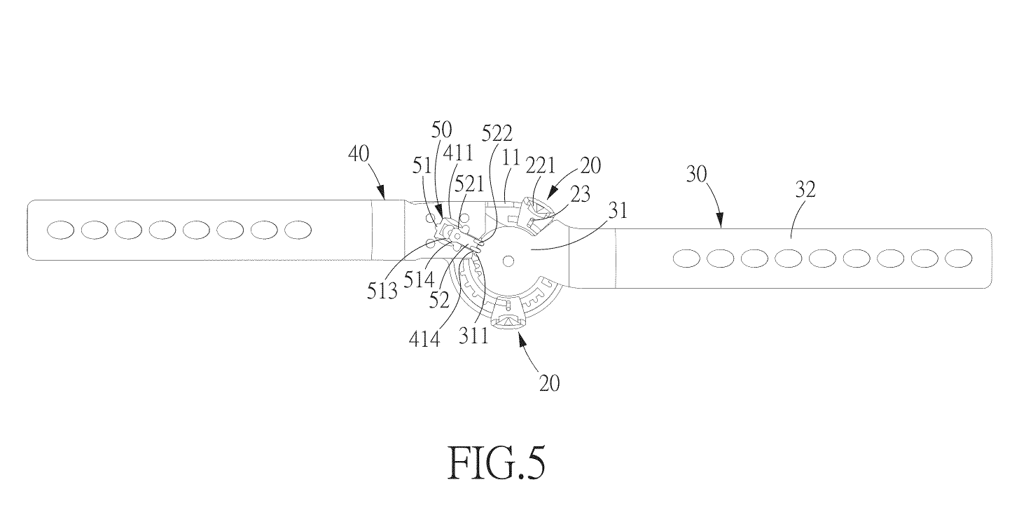

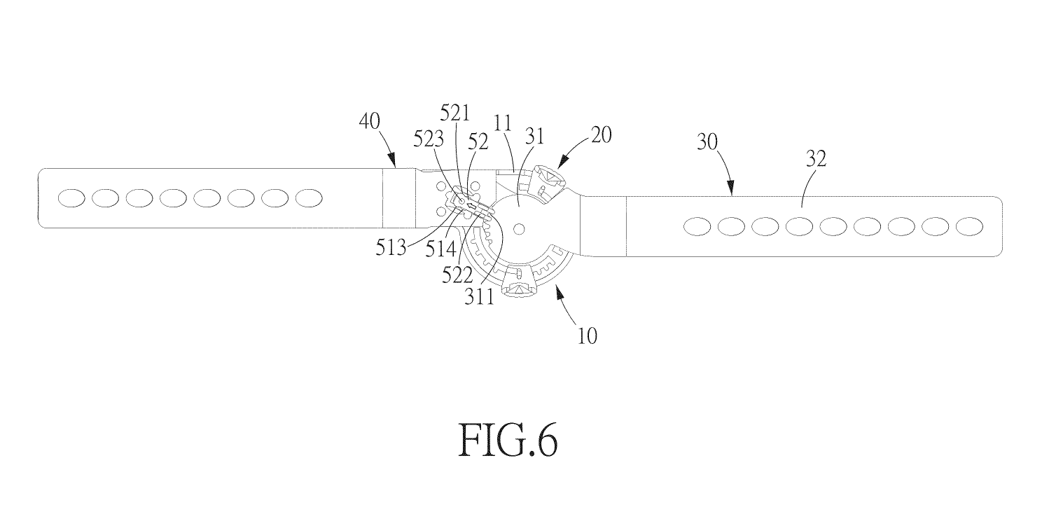

The angle-adjustable rehabilitation device of the present invention can be used to hold the limbs or restrict the angle of motion of the limbs. As shown in FIGS. 5 and 6, the angle-adjustable rehabilitation device of the present invention is used to hold the limbs in a fixed position, namely, preventing the limbs from arbitrary motion. In this fixed position, the engaging protrusion 521 of the control member 52 of the locking unit 50 is disposed in the second restricting notches 514 of the lubricating and positioning member 51, and the engaging teeth 522 of the control member 52 extend out of the open end 414 of the assembling slot 411 and engage with the positioning teeth 311 of the first rack 30.

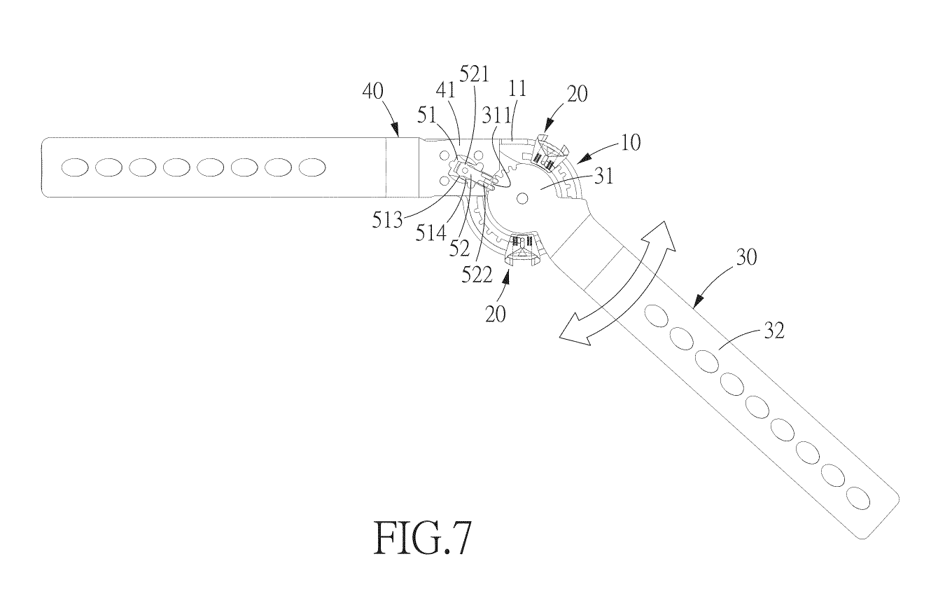

When the angle-adjustable rehabilitation device of the present invention is used to restrict the angle of motion of the wearer's limbs, as shown in FIGS. 6 and 7, the wearer can pull the control protrusion 523 that extends out of the cover member 11 to move along the operation slot 111, so as to drive the control member 52 to move away from the positioning teeth 311 of the first rack 30 until the engaging protrusion 521 of the control member 52 is engaged in the first restricting notches 513 of the lubricating and positioning member 51, so that the control member 52 is disengaged from the first rack 30. During the movement, the control member 52 slides against the lubricating and positioning member 51, and the lubricating and positioning member 51 provides lubrication when the control member 52 moves, which makes the rehabilitation device easy and convenient to use and control.

In this situation, the first rack 30 is allowed to pivot with respect to the two lateral covers 10 and the second rack 40, and the pivoting motion of the first rack 30 is restricted in the space between the two angle-adjustment positioning members 20. In this way, the angle-adjustable rehabilitation device of the present invention is capable of providing rehabilitation effect by restricting the angle of motion of the wearer's legs. Besides, the separators 13 of the lateral covers 10 serve as lubrication when the first rack 30 is rotating, allowing the first rack 30 to pivot more smoothly, and enabling the adjacent components to be abutted against one another more closely, to reduce shake and friction.

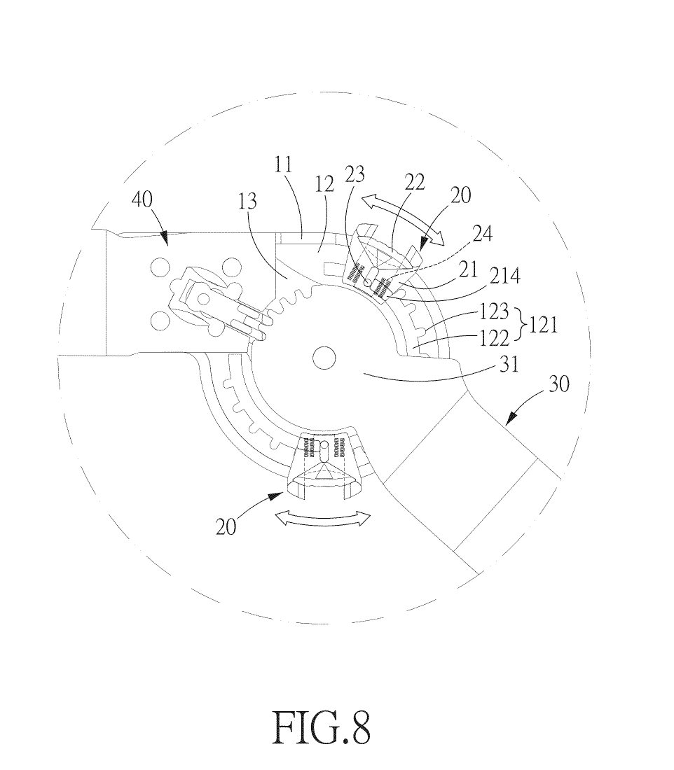

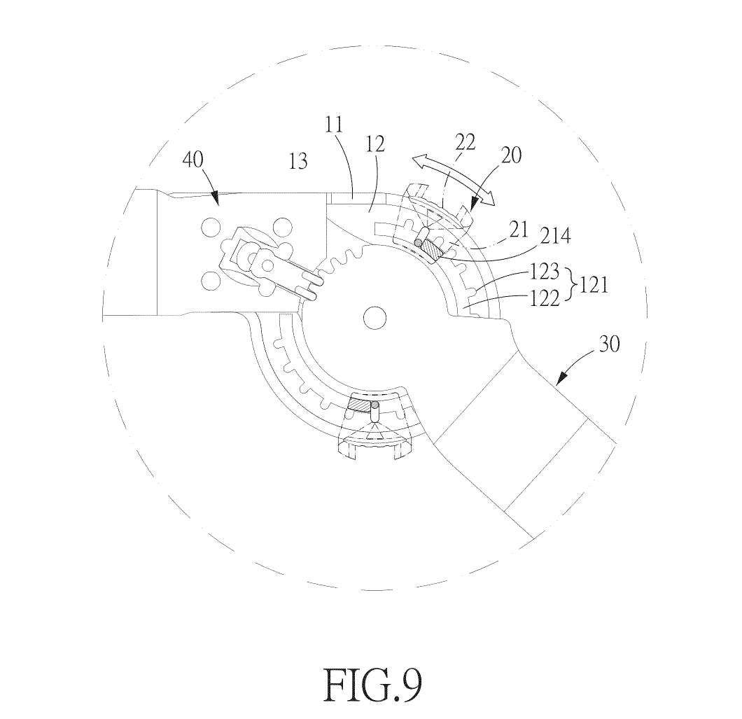

When the angle-adjustment positioning members 20 are not pressed, the springs 24 keep pushing the control members 22, and the connecting members 23 are engaged in the positioning notches 123 to maintain the angle-adjustment positioning members 20 in a fixed position. To adjust the angle of motion to be restricted, as shown in FIGS. 8 and 9, the wearer can press the control members 22 that extend out of the two lateral covers 10 to make the connecting members 23 disengage from the positioning notches 123, so that the slide blocks 214 of the main block 21 of the angle-adjustment positioning members 20 are allowed to slide along the slide section 122 of the toothed discs 12 to adjust the angle of motion to be restricted. During adjustment, the indication portion 222 of the retaining portions 221 points toward the arc-shaped angle scale 112 to make the angle of adjustment easily readable.

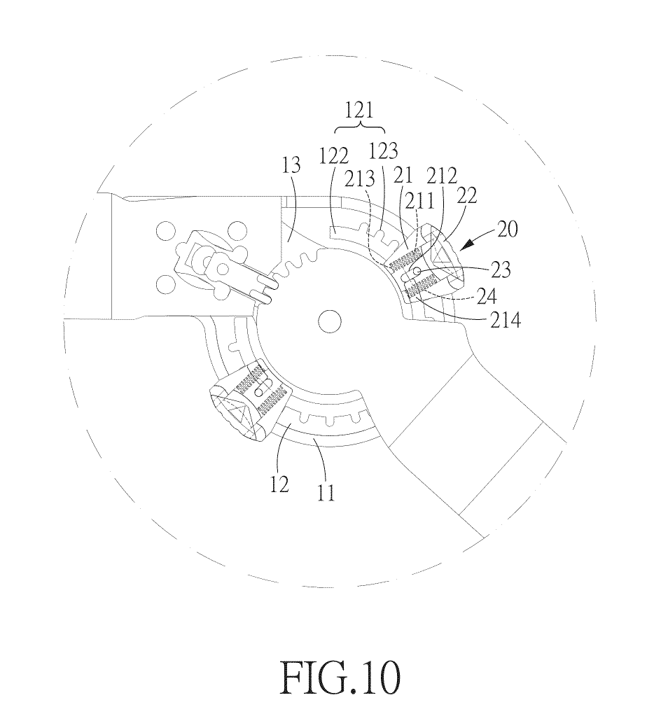

When the angle of motion to be restricted is adjusted, the wearer can release the control members 22, then the control members 22 are pushed back into the positioning notches 123 by the springs 24, so that the angle is adjusted and fixed, as shown in FIGS. 10-12. The angle-adjustable rehabilitation device of the present invention is very convenient to use since the wearer himself/herself can adjust the rotatable angle of the first rack 30 (which consequently restricts the angle of motion of the wearer's limbs) according to health condition, by synchronously pressing the two angle-adjustment positioning members 20.

It is clear from the above description that the locking unit 50 is capable of switching the first rack 30 between an adjustable position and a nonadjustable position. The lubricating and positioning member 51 provides lubrication when the control member 52 moves, which makes the rehabilitation device easy to use and control. When in the adjustable position, the rotatable angle of the first rack 30 can be freely adjusted. The soft separator 13 enables the adjacent components to be abutted against one another more closely, so as to reduce shake, friction and improve buffering effect, and also facilitate the angle adjustment. The control members 22 of the two angle-adjustment positioning members 20 protrude out of the two lateral covers 10, therefore, angle adjustment can be easily done without removing any components. Furthermore, the arc-shaped angle scale 112 on the two lateral covers 10 makes the angle adjustment more readable and controllable, and the first rack 30 can be moved back into the non-adjustable position automatically after angle adjustment is done.

While we have shown and described various embodiments in accordance with the present invention, it is clear to those skilled in the art that further embodiments may be made without departing from the scope of the present invention.

What is claimed is:

1. An angle-adjustable rehabilitation device comprising:
two lateral covers each including a cover member, each of the cover members including at least one angle-adjustment section which consists of a plurality of positioning notches defined on an arc-shaped slide section in a spaced manner, the positioning notches being in communication with the slide section, each of the toothed discs being provided with an elongated slot, the cover member of one of the two lateral covers being formed with an operation slot in communication with the elongated slot;
between the two lateral covers being disposed two angle-adjustment positioning members, a first rack and a second racks in such a manner that the two cover members are oppositely arranged to define therebetween a lateral slot;
a buffer means being disposed between the two lateral covers and the first and second racks to reduce friction, improve buffering effect and enable the two lateral covers and the first and second racks to be abutted more closely against one another;
each of the two angle-adjustment positioning members including a main block, a control member, a connecting member and two springs; wherein:
the main block is formed with a cavity and a slide groove in communication with the cavity;
the control member is partially disposed in the cavity;
the connecting member is interactively connected to the control member and extends out of the slide groove, each of the springs is disposed between the control member and a bottom of the cavity;
on each of two opposite outer surfaces of the main block is provided an arc-shaped slide block which is located between the cover members and slidably disposed in the slide section, the control member of each of the angle-adjustment positioning members partially extends out of the lateral slot, and the springs are pressed against the control members to maintain the connecting member at a position aligned to the positioning notches;
the first rack includes a first connecting section and a first extension section extending from the first connecting section, on the first connecting section is formed a plurality of positioning teeth, the first connecting section is pivotally disposed between the two lateral covers, and the first extension section extends out of the lateral slots;
the second rack is assembled to a locking unit and then disposed between the two lateral covers, the second rack includes a second connecting section and a second extension section extends from the second connecting section, the second connecting section is formed with an assembling slot; and
the locking unit includes an lubricating and positioning member and a control member; wherein:
the lubricating and positioning member are disposed the assembling slot, on an inner peripheral surface of the lubricating and positioning member are formed a first restricting notch and a second restricting notch;
the control member has one end formed with an engaging protrusion, and another formed with a plurality of engaging teeth, on one lateral surface of the control member is formed a control protrusion, the control member is abutted against the inner peripheral surface of the lubricating and positioning member in such a manner that the control protrusion of the control member is disposed in the first restricting notch or the second restricting notch of the elastic stop member, the lubricating and positioning member allows the control member to slide and to be fixed at different positions, the engaging teeth are engaged with the positioning teeth of the first rack, and the control protrusion is inserted through the elongated slot and extends out of the operation slot;
when the engaging protrusion is engaged in the first restricting notch, the engaging teeth of the control member are located in an open end of the assembling slot, and when the engaging protrusion is located within the second restricting notch, the engaging teeth of the control member extend out of the open end of the assembling slot and engage with the positioning teeth of the first rack.

2. The angle-adjustable rehabilitation device as claimed in claim 1, wherein the two lateral covers include a first lateral cover and a second lateral cover, the operation slot is formed on the cover member of the second lateral cover, and on an outer surface of the cover member of the second lateral cover is formed an arc-shaped angle scale.

3. The angle-adjustable rehabilitation device as claimed in claim 1, wherein the angle-adjustment section and the elongated slot are integrally formed on the inner surface of the cover member.

4. The angle-adjustable rehabilitation device as claimed in claim 1, wherein a toothed disc is provided on an inner surface of the cover member, and the angle-adjustment section and the elongated slot are provided on the toothed disc.

5. The angle-adjustable rehabilitation device as claimed in claim 1, wherein two said angle-adjustment sections are provided on the inner surface of the over member.

6. The angle-adjustable rehabilitation device as claimed in claim 1, wherein a part of the control member protrudes out of the cavity and extends laterally in opposite directions to form two L-shaped retaining portions, the retaining portions are located outside the lateral covers and abutted against the cover members, and on each of the retaining portions is formed an indication portion which is a triangular structure.

7. The angle-adjustable rehabilitation device as claimed in claim 1, wherein the angle-adjustment section and the elongated slot are integrally formed on the inner surface of the cover member, the flexible member is a separator which is made of a material softer than the first and second racks, the cover members and the toothed disc, the first and second racks, the cover members and the toothed disc are made of metal, and the separator is made of plastic or rubber, the separator is disposed between the cover members and the first and second racks.

8. The angle-adjustable rehabilitation device as claimed in claim 6, wherein a toothed disc is provided on an inner surface of the cover member, the angle-adjustment section and the elongated slot are provided on the toothed disc, the flexible member is a separator which is made of a material softer than the first and second racks, the cover members and the toothed disc, the first and second racks, the cover members and the toothed disc are made of metal, and the separator is made of plastic or rubber, the separator is disposed between the cover members and the first and second racks.

9. The angle-adjustable rehabilitation device as claimed in claim 1, wherein the control protrusion is cylindrical.

10. The angle-adjustable rehabilitation device as claimed in claim 1, wherein the assembling slot includes a close end, two opposite walls and an open end, at the close end is formed a first positioning notch, and on the opposite walls are formed two second positioning notches, and an outer peripheral surface of the lubricating and positioning member is formed in the shape of the assembling slot, so as to make the lubricating and positioning member fit in the assembling slot.
